(19) United States
(12) Patent Application Publication
(10) Pub. No.: US 2010/0100093 A1
(43) Pub. Date: Apr. 22, 2010

(54) SYSTEM AND METHOD FOR CONTROLLED TISSUE HEATING FOR DESTRUCTION OF CANCEROUS CELLS

(75) Inventor: Larry Azure, La Conner, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: LaZure Technologies, LLC., La Conner, WA (US)

(21) Appl. No.: 12/561,242

(22) Filed: Sep. 16, 2009

Related U.S. Application Data
(60) Provisional application No. 61/097,218, filed on Sep. 16, 2008.

(51) Int. Cl. A61B 18/14; A61B 18/12

(52) U.S. Cl. 606/34; 606/41

(57) ABSTRACT

Methods and systems for delivering electrical energy for controlled heating or hyperthermia to a target tissue of a patient for destruction of cancerous cells or tissue.

### CROSS-REFERENCE TO RELATED APPLICATION

The present invention claims the benefit of priority under 35 U.S.C. §119(e) of U.S. Provisional Application No. 61/097,218, filed Sep. 16, 2008 (Attorney Docket No. 26533A-001600US), the entire content of which is incorporated herein by reference.

### BACKGROUND OF THE INVENTION

The present invention relates generally to electric field delivery to a tissue of a patient. More particularly, the present invention provides systems, devices and related methods for electric fields delivery for precisely controllable tissue heating and/or preferential destruction of cancerous cells and tissue ablation.

Tissue heating for cancer tissue hyperthermia includes treatment in which the temperature of either local tissue or the whole body is raised to a therapeutic level for the destruction of tumors. Cancer hyperthermia has been studied for the last several decades, with research often focusing on the combined effects of hyperthermia on cells and other treatments such as ionizing radiation therapy and chemotherapy.

While study results provide promising evidence and rationale supporting application of hyperthermia in cancer treatment, implementation remains difficult. Perhaps the most significant obstacle for practical application of hyperthermia is the generation and accurate control of heating to tumor tissues. Effective temperature ranges are narrow, with excessive temperatures indiscriminately destroying both healthy tissue and tumor tissue alike, and insufficient heating or low temperatures having minimal or no effect. Conventional existing methods for whole body heating include, for example, hot wax, hot air, hot water, fluid perfusion, RF fields and microwaves. However, existing equipment and methodologies have so far been inadequate in delivering accurate and controlled heating to tissues in more optimal temperature ranges, particularly to sub-surface or deep-seated tissues.

Accordingly, there is a continuing interest to develop devices and methods for accurate and controlled heating of tumor tissues and tissues including cancerous cells.

### BRIEF SUMMARY OF THE INVENTION

The present invention provides systems, devices and related methods for applying electric fields, which can be delivered for precisely controllable tissue heating and/or preferential destruction of cancerous cells and tissue ablation. Methods and devices of the present invention will generally be designed to advance an electrode or plurality of electrodes to a target tissue region and apply an electric field to the target tissue region. The electrode or plurality thereof can be positioned such that the applied electric field extends or radiates through the target tissue region, including, for example, where the electric field radiates outwardly and/or in a plurality of directions, e.g., radially, through the target tissue. Energy application can be selected so as to deliver mild and controlled heating of the target tissue to a desired temperature or range.

For a fuller understanding of the nature and advantages of the present invention, reference should be made to the ensuing detailed description and accompanying drawings. Other aspects, objects and advantages of the invention will be apparent from the drawings and detailed description that follows.

### BRIEF DESCRIPTION OF THE DRAWINGS

FIG. 1 illustrates a device according to an embodiment of the present invention.

FIGS. 2A through 2D illustrate a device according to another embodiment of the present invention.

FIGS. 2E through 2H illustrate a device having a deployable microtube and electrode configuration, according to another embodiment of the present invention.

FIG. 2I illustrates a probe device according to another embodiment of the present invention.

FIGS. 3A through 3D illustrate field delivery in a target tissue according to various embodiments of the present invention.

FIGS. 4A and 4B illustrate a system for delivery of electric fields to a tissue of a patient using a plurality or array of electrodes.

FIG. 5 includes a flowchart illustrating a method according to an embodiment of the present invention.

FIG. 6 includes a diagram illustrating a system according to an embodiment of the present invention.

### DETAILED DESCRIPTION OF THE INVENTION

The present invention includes systems, methods and devices for applying electric fields to a target tissue for controllable tissue heating and/or preferential destruction of cancerous cells and tissue ablation.

Energy application and delivery of controlled mild tissue heating or hyperthermia can offer several advantages. First, energy delivery according to the present invention further advantageously allows a more controlled or precise therapeutic energy dose both in terms of delivery of the desired current and resulting hyperthermia effects, as well as more accurate delivery to the target or intended tissue. Current flow can be established between electrodes in a bipolar arrangement, with current flow established and substantially contained between the spaced electrodes. Tissue heating can be more precisely controlled to prevent or minimize excessive heating and/or hot spots that can cause unintended damage to healthy or non-target tissues. For example, energy delivery can be selected (e.g., frequency ranges between about 50 kHz to about 300 kHz) such that tissue heating occurs significantly, and in some cases predominately, due to tissue resistance, rather than the high-frictional heating observed at high frequencies (e.g., 500 kHz or greater), the latter of which can include significant tissue temperature gradients throughout the treated tissue, with significant tissue temperature changes occurring through a volume of treated tissue as a function of electrode distance. While heating may occur due to both tissue resistance and frictional heating, with relative reduction of high friction type heating a more constant and controlled heating between opposing electrodes may be delivered.

Another advantage of the present inventive methods and systems is that energy delivery and application of mild hyperthermia as described has been observed to be surprisingly effective in preferentially damaging and destroying cancerous cells compared to non-cancerous or healthy cells/tissue. Preferential destruction, as described herein, refers to establishing current flow as described with application of hyperthermia, generally below about 50 degrees C., such that cytotoxic effects of treatment are, on average or as a whole, more destructive and/or lethal to cancerous or hyperplastic cells (e.g., cells exhibiting or predisposed to exhibiting unregulated growth) compared to non-cancerous or healthy cells. In some instances, establishing current flow and induction of mild hyperthermia as described herein is remarkably effective in preferentially destroying cancerous cells with limited or no observable damage to non-cancerous tissues.

Furthermore, and without being bound by any particular theory, electrode configuration and field application as described in certain embodiments (e.g., radially and/or in a plurality of different directions) may take advantage of tumor or mitotic cell physiology to increase treatment effectiveness, and can include a more optimal or effective orientation of the applied field with respect to dividing cells of the target region. For example, energy application can be accomplished such that current fields are substantially aligned at some point during energy delivery with division axes of dividing cells (e.g., cancerous cells), thereby more effectively disrupting cellular processes or mitotic events (e.g., mitotic spindle formation and the like). As cancerous cells are dividing at a higher rate compared to non-cancerous cells, field application in this manner may preferentially damage cancerous cells compared to healthy or non-dividing cells. It will be recognized, however, that energy application according to the present invention likely has several or numerous cytotoxic effects on cells of the target region and that such effects may be cumulatively or synergistically disruptive to a target cell, particularly to cells disposed or pre-disposed to unregulated growth (i.e., cancerous cells). Other cytotoxic or disruptive effects of the energy application as describe herein may occur due, for example, to application of mild hyperthermia (e.g., mild heating of tissue between about 40 to 48 degrees C.; or less than about 50 degrees C.); ion disruption, disruption of membrane stability, integrity or function; disruption of cellular components and/or organelles; and the like.

Various electrode or probe configurations can be utilized according to the present invention. In one embodiment, electrodes can include an array of needle electrodes, which can be fixed to common support (e.g., housing) or separately positionable and controlled. Such a plurality or array of electrodes can include a straight-needle array including electrically conductive material such as stainless steel, gold, silver, etc. or combination thereof. An array of straight-needle electrodes can be coupled to a rigid needle support or housing that can ensure correct positioning of each individual needle relative to the others. The needles can be arranged parallel to one another with opposing rows and/or columns of electrodes ensuring the field is delivered to and contained within the target area. Needle length and needle spacing can vary depending on the actual dimensions of the target tissue. Individual needle placement can be guided using imaging (e.g., ultrasound, X-ray, etc.) and relative needle position can be maintained with a rigid grid support (e.g., housing, template, etc.) that remains outside the body. The needle assembly will electrically connect to the control system or module, e.g., via insulated wires and stainless steel couplings.

In another embodiment, a probe can include one or more electrodes that are deployable from an elongate probe housing or catheter. Such embodiments may be particularly useful for treatment of target areas more difficult to access with an array of fixed needles. Such deployable type probes, and others described herein, can be inserted percutaneously through the skin of the patient and into the target tissue, or advanced through a body lumen. As above, appropriate imaging technology can be used to guide the precise placement of the probe in the target site. In one embodiment, a deployable type probe can include outer polyurethane sheath housing pre-shaped deployable shape memory metal tines and a stainless steel central electrode tip. Conductive surfaces can further be coated with a highly conductive material.

Another embodiment of the probe can include one or more expandable elements (e.g., balloon) that can be individually positioned around a target area or organ, or advanced in a body lumen, and then deployed and “inflated” to achieve maximum surface area and optimal distribution of the therapeutic field. In one example, an electrically active segment of the expandable element can include an electrically conductive material (e.g., silver, gold, etc.) coated or deposited on a mylar balloon. Prior to deployment and inflation, the expandable element can be contained inside a flexible catheter that can be guided to the treatment area. Once the delivery catheter is positioned, the “balloon” can be deployed and expanded via the circulation of fluid through the balloon, which can have a selected or controlled temperature and may act as a heat sink. The therapeutic field can than be delivered via the silver coating on the mylar balloon. Two or more probes deployed in this fashion will serve to contain the field within the treatment area.

Electrodes and probes of the present invention can be coupled to control system or control module designed to generate, deliver, monitor and control the characteristics of the applied field within the specified treatment parameters. In one embodiment, a control system includes a power source, an alternating current (AC) inverter, a signal generator, a signal amplifier, an oscilloscope, an operator interface and/or monitor and a central processing unit (CPU). The control unit can manually, automatically, or by computer programming or control, monitor, and/or display various processes and parameters of the energy application through electrodes and to the target tissue of the patient. While the control system and power source can include various possible frequency ranges, current frequency delivered to target tissue will be less than about 300 kHz, and typically about 50 kHz to about 250 kHz (e.g., 100 kHz). Frequencies in this range have been observed as effective in precisely controlling the energy application to the target tissue, controlling thermal effects primarily to mild thermal application, and preferentially destroying cancerous cells with limited or no observable damage to non-cancerous tissues.

Energy application according to the present invention can further include mild or low levels of hyperthermia. In some embodiments, small changes/elevations in temperature in the target tissue region may occur, but will typically be no more than about 10 degrees C. above body temperature, and may be about 2 degrees to less than about 10 degrees C. above body temperature (e.g., normal human body temperature of about 38 degrees C.). Thus, local tissue temperatures (e.g., average tissue temperature in a volume of treated tissue) during treatment will typically be less than about 50 degrees C., and typically within a range of about 40-48 degrees C. In one embodiment, average target tissue temperature will be selected at about 42-45 degrees C. As target tissue temperatures rise above about 40-42 degrees C. during treatment, the cytotoxic effects of energy delivery on cancerous cells of the target region are observably enhanced, possibly due to an additive and/or synergistic effect of current field and hyperthermic effects. Where mild hyperthermic effects are substantially maintained below about 48 degrees C., the energy delivery according to the present invention appears to more preferentially destroy cancerous cells compared to healthy or non-cancerous cells of the target tissue region. Where energy delivery induces tissue heating substantially in excess of about 45-48 degrees C. (e.g., particularly above 48-50 degrees C.), the preferential cytotoxic effects on cancerous cells may begin to diminish, with more indiscriminate destruction of cancerous and non-cancerous cells occurring. Thus, a significant advantage of treatment methods according to the present invention includes the ability to precisely and accurately control energy delivery and induced hyperthermic effects, such that tissue hyperthermia can be accurately controlled and maintained in a desired temperature range(s)—e.g., temperature ranges selected for more targeted or preferential destruction of cancerous cells compared to non-cancerous cells.

Tissue temperatures can be selected or controlled in several ways. In one embodiment, tissue temperatures can be controlled based on estimated or known characteristics of the target tissue, such as tissue impedance and tissue volume, blood flow or perfusion characteristics, and the like, with energy application to the tissue selected to deliver an approximated controlled mild increase in tissue temperature. In another embodiment, tissue temperature can be actively detected or monitored, e.g., by use of a feedback unit, during treatment, with temperature measurements providing feedback control of energy delivery in order to maintain a desired target tissue temperature or range. Temperature control measures can include electronics, programming, thermosensors and the like, coupled with or included in a control unit or module of a system of the invention. Further, use of inflatable/expandable balloons and circulation heated/cooled inflation media further facilitates control and delivery of the desired treatment temperature to the target tissue.

Energy application and induction of hyperthermia in a target tissue region according to the present application can include delivery of various types of energy delivery. As described, application of generally intermediate frequency range (e.g., less than about 300 kHz) alternating current in the RF range has been observed as effective in establishing mild heating and hyperthermia, as well as current fields in a controlled manner so as to provide a cytotoxic effect, and in some instances, a preferential destructive effect to cancerous cells of a target tissue volume/region. It will be recognized, however, that additional energy applications and/or ranges may be suitable for use according to the present invention, and that systems and methods of the present invention may be amenable to use with other or additional energy applications. For example, energy application can include current flow having frequencies found generally in the RF range, as well as microwave range, including higher frequencies such as 300-500 kHz and above, and may further be amenable to use with direct current applications. Applied current can be pulsed and/or continuously applied, and energy delivery can be coupled with a feedback-type system (e.g., thermocouple positioned in the target tissue) to maintain energy application and/or tissue heating in a desired range.

In certain embodiments, particularly where energy application is selected for lower power delivery/ablation, the control system can be designed to be battery powered and is typically isolated from ground. AC current is derived from the integrated power inverter. An intermediate frequency (e.g., less than 300 kHz; or about 50 kHz to about 250 kHz) alternating current, sinusoidal waveform signal is produced from the signal generator. The signal is then amplified, in one non-limiting example to a current range of 5 mA to 50 mA and voltage of up to 20 Vrms per zone. Field characteristics including waveform, frequency, current and voltage are monitored by an integrated oscilloscope. Scope readings are displayed on the operator interface monitor. An integrated CPU monitors overall system power consumption and availability and controls the output of the signal generator and amplifier based on the treatment parameters input by the operator. The operator can define treatment parameters to include maximum voltage, maximum current or temperature, maximum power, and the like.

Imaging systems and devices can be included in the methods and systems of the present invention. For example, the target tissue region can be identified and/or characterized using conventional imaging methods such as ultrasound, computed tomography (CT) scanning, X-ray imaging, nuclear imaging, magnetic resonance imaging (MRI), electromagnetic imaging, and the like. In some embodiments, characteristics of the tumor, including those identified using imaging methods, can also be used in selecting ablation parameters, such as energy application as well as the shape and/or geometry of the electrodes. Additionally, these or other known imaging systems can be used for positioning and placement of the devices and/or electrodes in a patient's tissues.

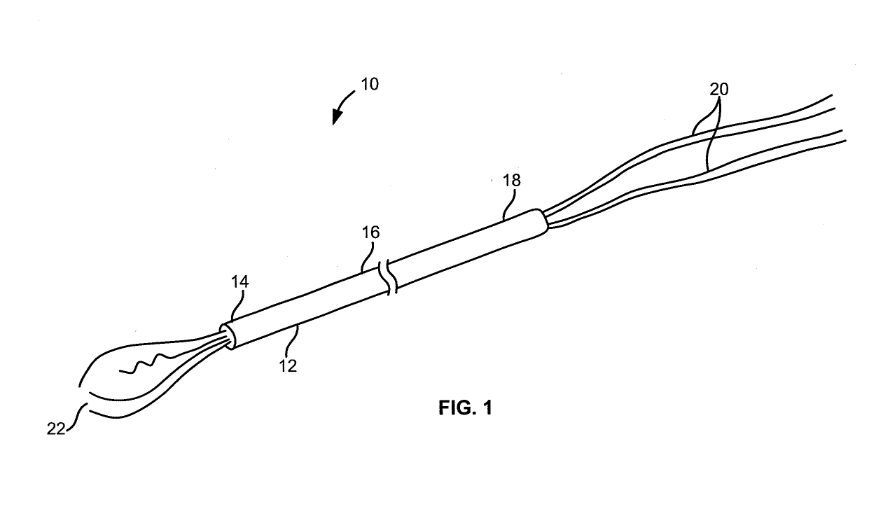

Referring to FIG. 1, a device according to an embodiment of the present invention is described. The device 10 includes a delivery member 12 having a distal portion 14 and a proximal portion 16. The device 10 further includes a proximal portion 18 of the device that can be coupled (e.g., removably coupled) to the delivery member 12. Additionally, the device 10 can include conductive cables 20 electrically coupled to an energy source (not shown). The device includes a plurality of electrodes 22 at the distal portion 14 of the delivery member 12. The electrodes 22 can be positioned or fixed, for example, at the distal end of the delivery member 12 or positionable and deployable from a lumen of the delivery member 12 and retractable in and out of the distal end of the delivery member 12. The electrodes 22 can include a non-deployed state, where the electrodes 22 can be positioned within a lumen of the delivery member 12, and a deployed state when advanced from the distal end of the delivery member 12. Electrodes 22 are advanced out the distal end and distended into a deployed state substantially defining an ablation volume.

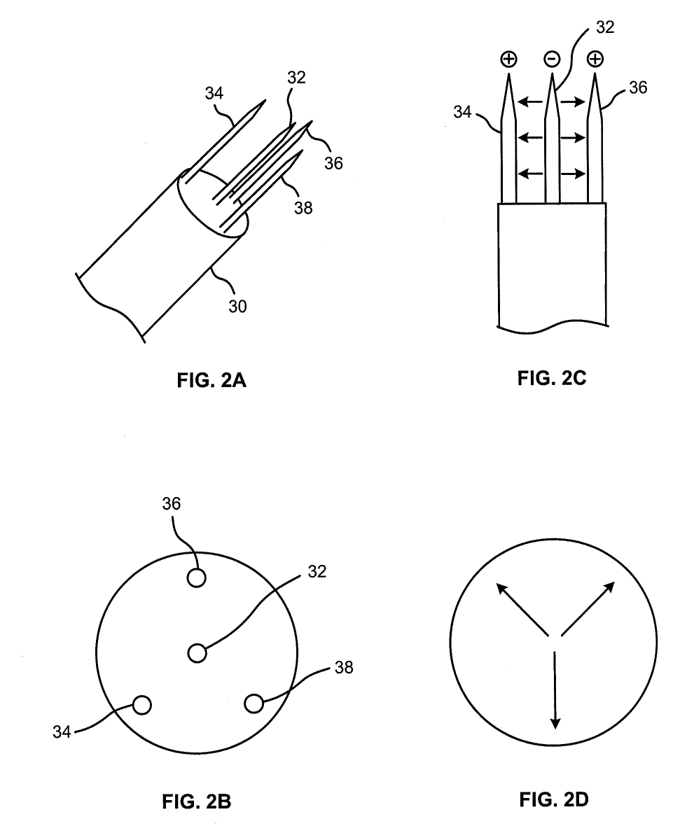

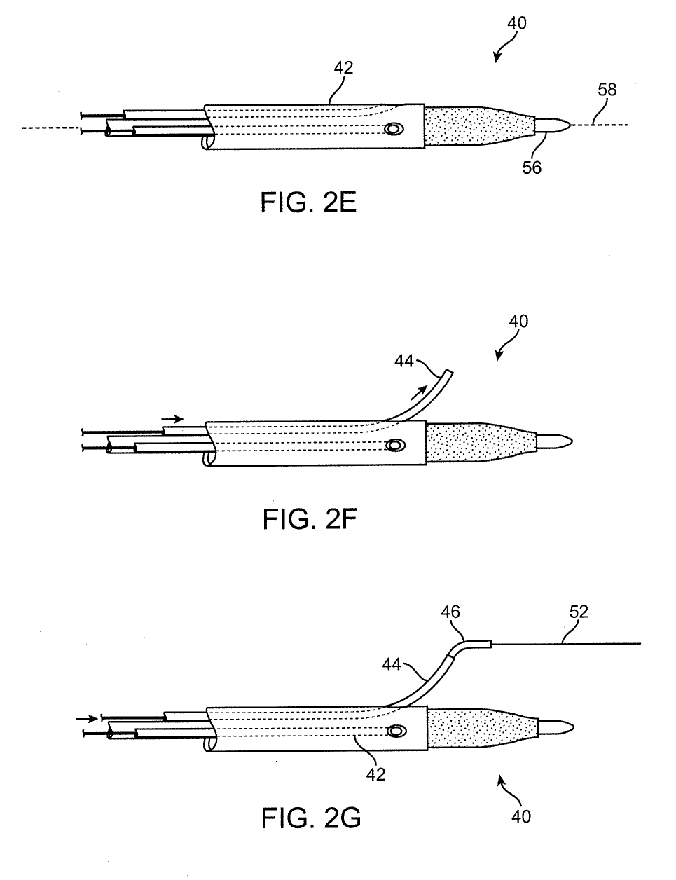

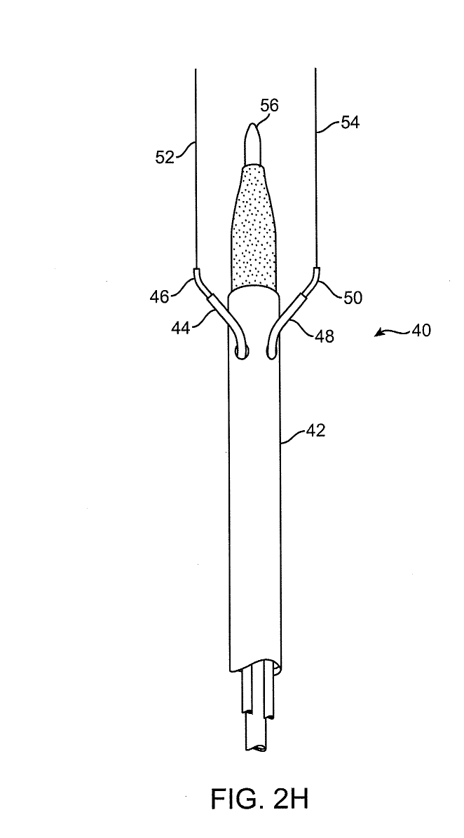

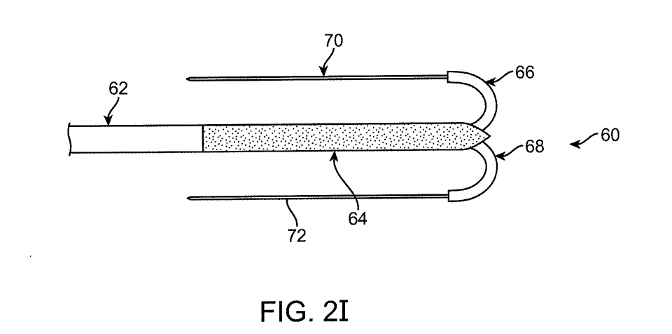

In another embodiment, a probe can include a plurality of needle electrodes fixed to or positioned on a body or housing of a device. FIGS. 2A through 2C show a device having a plurality of electrodes coupled to a housing, according to another embodiment of the present invention. As shown, the device 30 includes a plurality of electrodes extending from the distal portion (e.g., housing) of the device. FIG. 2A shows a three dimensional side view of the device having the plurality of electrodes. FIG. 2B shows a top view of the device illustrating the electrode arrangement. The plurality includes a centrally positioned electrode 32 and outer electrodes 34, 36, 38 spaced laterally from the central electrode 32. The illustrated electrodes include substantially linear needle-like portions or needle electrodes. The electrodes extend from the distal portion of the device and are oriented to be substantially parallel with the longitudinal axis of the device 30. Additionally, each electrode is substantially parallel with other electrodes of the plurality. The plurality of electrodes substantially define the ablation volume, with the outer electrodes 34, 36, 38 substantially defining a periphery of the ablation volume and the electrode 32 positioned within or at about the center point of the defined periphery. Each of the electrodes can play different roles in the ablation process. For example, there can be changes in polarity and/or polarity shifting between the different electrodes of the device. As with other devices of the invention, electrodes can be electrically independent and separately addressable electrically, or two or more electrodes can be electrically connected, for example, to effectively function as one unit. In one embodiment, for example, outer electrodes 34, 36, 38 can be electrically connected and, in operation, include a polarity different from that of the inner electrode 32. As illustrated in FIG. 2C the electrodes 32 and 34, 36 of the device can include opposing charges (e.g., bipolar). In such an instance, the applied electrical current can provide an electrical field, as illustrated by the arrows, extending radially outward from the central electrode 32 and toward the peripherally positioned or outer electrode(s) 34, 36. FIG. 2D illustrates the concept of a current flow center, where current flow is established through about a center location of a treatment volume.

In some embodiments, electrodes can be deployable from small, electrode guides or positioning tubes, e.g., microtubes or microcatheters, positionable in and advanceable from a distal portion of an ablation probe. The terms catheter or microcatheter, as used herein, refer generally to an elongate tube having a lumen. For example, an ablation probe of the present invention can include a distal portion or a delivery member having a lumen with electrode aiming/positioning microtubes/microcatheters positioned within the lumen of the delivery member, with electrodes disposed in the microcatheters and deployable therefrom. Both microcatheters and electrodes can include a shape memory metal and include a preformed shape for deployment. In use, the distal portion of the probe can be positioned proximate to a target tissue, for example, by advancing the probe through a patient's tissue. Once in position, a microcatheter can be deployed from the delivery member and can act as an initial advancement or guide tube as advanced or deployed from the delivery member for initial aiming and/or positioning of the electrode disposed therein. Following advancement and positioning of the microcatheter, the electrode can be deployed from the microcatheter for desired positioning of the electrode at or in the target tissue region. In another embodiment, an assembly of two or more concentrically positioned microtubes may be utilized for a sort of telescoping-like advancement/deployment. Such a configuration can include a first microtube that advances in a first direction, and a second microtube deployable from the lumen of the first microtube and advancing in a second direction, and an electrode disposed in the lumen of the second microtube and deployable therefrom. The described “multi-phase” type of microcathter/electrode deployment configuration can in some instances provide more versatility and improved functionality in positioning of electrodes, and can permit a wider range of motion or positioning of an electrode in a tissue compared to other configurations, such as deployment of only an electrode alone. The described configuration was found to be well suited, for example, for positioning of outer electrodes (e.g., secondary electrodes) to define an ablation volume, particularly where a microtube and electrode are first advanced in a direction including an advancement portion angling away from the delivery member, delivery member long axis, or current flow center and then advanced in a direction that moves the electrode back toward the delivery member/center and/or along a path substantially parallel to the delivery member or long axis thereof. More complex tissue penetration paths, such as a sort of “S” shaped path, can more effectively be accomplished using microtube assemblies as described. Use of one or more microtubes as described can further advantageously limit tissue damage (e.g., slicing of the tissue) that can occur, e.g., from lateral force of an electrode on the patient's tissue during electrode deployment, where a non-uniform tissue penetration path is attempted with a single electrode having a sharper angle bend or non-uniform bend or configuration—e.g., a single electrode having an “S” shaped configuration.

An ablation probe having deployable electrodes and microcatheters is described with reference to FIGS. 2E through 2H. The probe 40 includes a distal portion including a delivery member 42 having a lumen, and microcatheters 44, 46, 48, 50 with electrodes 52, 54. The probe 40 further includes distally positioned electrode 56, which can be substantially fixed or deployable. FIG. 2E illustrates probe 40 in a non-deployed state. FIG. 2H illustrates the probe 40 in a deployed state or configuration. For illustrative and reference purposes a long axis 58 of the probe 40 is shown in FIG. 2E. Microcatheters are deployed from the delivery member, as illustrated in FIGS. 2F and 2G. FIG. 2F shows deployment of a first microcatheter 44 along a first direction or path. Deployment of microcatheter 44 can include application of a force to a proximal portion of the microcatheter 44 so as to advance the distal portion of the microcatheter 44 from the delivery member 42 for deployment and initial positioning or aiming. Deploying the microcatheter 44 from the delivery member 42 guides the microcather 44 along a guide path (e.g., tissue penetration path) that can angle or curve in one or more desired directions or angles. FIG. 2G shows deployment of a second microcatheter 46 from the lumen of the first microcatheter 44, and deployment of electrode 52 from a lumen of the second microcather 46. Thus, electrode 52 can be deployed from the microcatheter 46, e.g., in a different direction than the first microcatheter 44, for further positioning, as illustrated in FIG. 2G. In this manner, the advancing the first microcatheter 44 first guides deployment in a first direction, e.g., outward or away from the delivery member 42. Deployment of the second microcatheter 46 advances along a second path or direction, e.g., inward or a path angling in a direction along or substantially parallel to the long axis 58 of the delivery member. Deploying the electrode 52 advances the electrode 52 in a third direction, e.g., further along a path substantially parallel to the delivery member 42 or long axis thereof 58. In use, the electrode 52, 54 at least partially defines the outer portion or perimeter of the ablation volume, with electrode 56 positioned near or at about the center of the volume, permitting current flow extending within the volume and between electrodes 52, 54 and 56.

Probes including microcatheter/electrode assemblies can include various designs and/or configurations. FIG. 2I illustrates a probe 60 having microcatheter/electrode assembly, according to another embodiment of the present invention. Probe includes a delivery member having a non-electrically active portion 62 (e.g., insulated portion) and an electrically active portion 64 that can function as an inner or central electrode. The probe 60 further includes microcatheters 66, 68 deployable from a distal portion of the delivery member. Electrodes 70 and 72 are deployable from microcatheters 66 and 68, respectively. Microcatheters 66, 68 can include a circular shape or uniform arch so as to advance from the delivery member distally and curving back proximally relative to the delivery member. Electrodes 70, 72 can include a substantially straight or linear configuration so as to advance from the microcatheters and proximally, and substantially parallel to the delivery member or long axis thereof. Energy delivery can include establishing current flow between electrode 64 and electrodes 70, 72, the latter substantially defining an ablation volume.

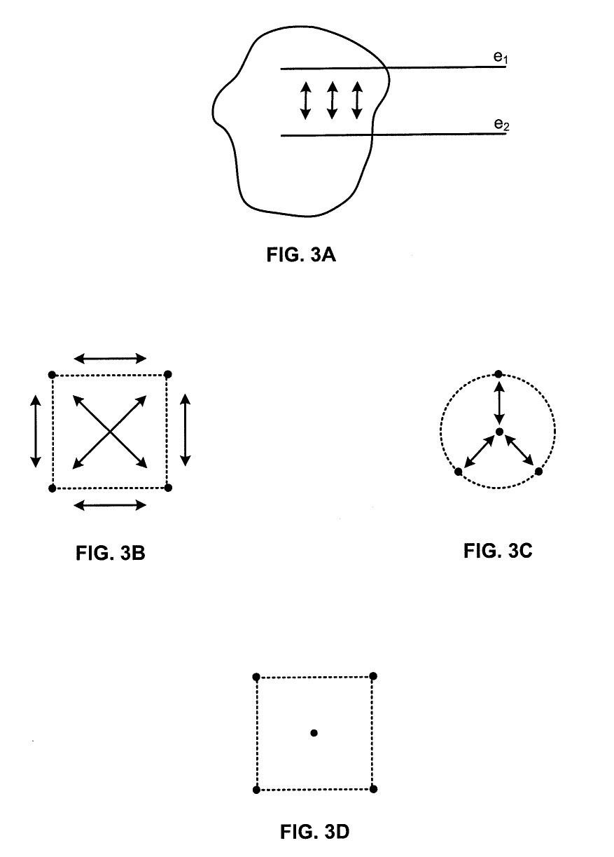

Energy delivery between positioned electrodes is further described with reference to FIGS. 3A through 3C. Electrodes can be positioned in a target tissue and activated in pairs or groups such that the desired electric field is delivered to the target tissue between the electrodes and, in some instances, in a radial orientation or in a plurality of different directions. FIG. 3A conceptually illustrates establishment of a current field with two spaced electrode elements (e₁ and e₂) as a basic field delivery unit according to an embodiment of the present invention. As shown, distal portions of two electrodes (e₁ and e₂) of a plurality positioned in a target tissue and activated as an electrode pair or circuit, with the applied current substantially contained between the two. Thus, electrodes can be activated in a bipolar configuration, with current flowing between electrodes (e.g., between e₁ and e₂) and the tissue between the electrodes acting as a flow medium or current pathway between the electrodes. Positioning and activation of pairs or relatively small groups of electrodes in this manner allows more precise control of the current applied to the tissue, containment of the applied field to the desired location, as well control of heating or limited temperature increase in the target tissue. Several factors may lend to improved control of therapeutic effects of the delivered fields according to the present invention. First, as discussed above activating electrode in a bipolar configuration or so as to form a circuit allows the applied field to substantially be contained within the volume defined by the positioned electrodes. Second, energy delivery can be selected (e.g., frequency ranges between about 50 kHz to about 300 kHz) such that tissue heating occurs predominately due to tissue resistance, rather than the high levels of frictional heating observed at high frequencies (e.g., 500 kHz or greater). High frequency/high friction type heating is typically characterized by significant tissue temperature gradients throughout the treated tissue, with substantially higher tissue temperatures occurring near the electrode. Where high friction type heating is reduced relative to heating occurring due to tissue resistance, a more constant and controlled heating between opposing electrodes can be delivered.

In some embodiments of therapeutic energy delivery according to the present invention, electrode positioning and/or device configuration advantageously allows delivery of field throughout a target tissue volume in a plurality of different directions, such as radial field orientation and application through the target volume. FIGS. 3B through 3D illustrate simplified plan views of electrode positioning and spacing for field application according to exemplary embodiments of the present invention. As shown in FIG. 3B, a simple four electrode grouping can be selected for use in treatment, with an applied field established between groups or pairs (e.g., different opposing electrode pairs). Groups or pairs of different electrodes can be differentially activated for field application in different directions/orientations. Electrode positioning can further include outer electrodes substantially defining a volume, and an electrode positioned within the volume. Electrode activation can include application of current flowing between a centrally positioned electrode and outer or secondary electrodes positioned spaced from the inner or center electrode. Thus, an exemplary delivery unit can include an inner or centrally located electrode surrounded by spaced electrodes, with the applied field extending between the central electrode and the outer spaced electrodes. In this manner, the outer electrodes can essentially define an ablation volume with the inner/central electrode positioned within the volume. Field delivery in this way is advantageously controlled and substantially contained within the ablation volume. Furthermore, field delivery in this manner advantageously allows a current field to be established with current flow in a radial and plurality of different directions through the treatment volume, e.g., extending through or from a flow center located about the centrally positioned electrode. FIG. 3C illustrates exemplary electrode positioning including outer electrodes and an inner or centrally located electrode, for defining a discrete target tissue volume for treatment and application of treatment filed extending radially through the volume. Electrode positioning will not be limited to any particular configuration, and various arrangements will be possible.

In another embodiment of the present invention, systems and methods can include a plurality of electrodes (e.g., needle electrodes) that can be individually advanced and positioned in the target tissue, and electrically activated for energy delivery. In such an embodiment, an array of electrodes can be advanced through the tissue of the patient and electrically activated (e.g., differentially activated) to deliver current field in a plurality of different directions. An array or plurality as described can include various numbers of electrodes, and the selected number can depend, at least partially, on factors such as target tissue characteristics, treatment region, needle size, and the like. An array can include a few to several dozen electrodes. In one example, an array can include about a few electrodes, to about a dozen or hundred, or more (e.g., 10-100, 5-200, any number therebetween, or more) electrodes for positioning in the target tissue region.

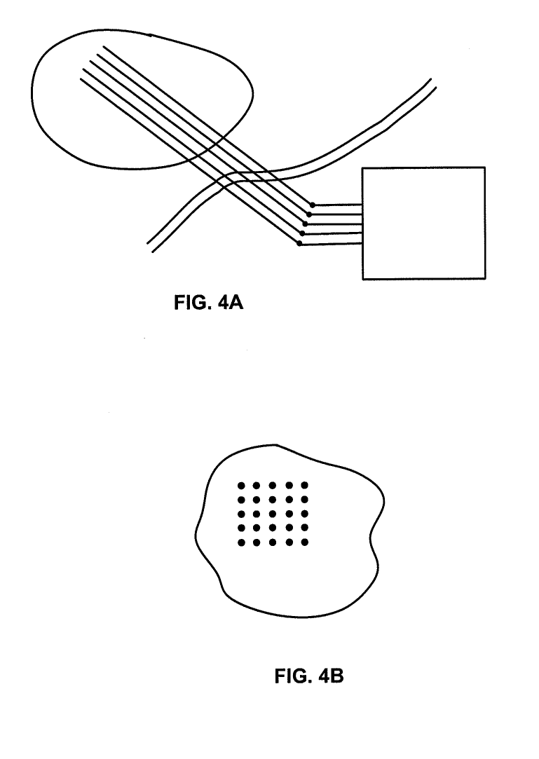

A system and method for delivering electric fields according to the present invention is described with reference to FIGS. 4A and 4B. The system includes a plurality of individual needle electrodes that can be positioned in a target tissue. Elongated needle electrodes will include a distal portion and a proximal portion. The proximal portion of each electrode will be electrically connected to a system control unit or module, which includes electronics, storage media, programming, etc., as well as a power generator, for controlled delivery of selected electrical fields to the target tissue. In use, a plurality of electrodes will be advanced through the tissue and to a desired position, as shown in FIG. 4A. Electrode positioning can include, for example, insertion and advancement through the skin and through the tissue of the patient. Electrode positioning and arrangement within the target tissue can be precisely controlled and may occur under the guidance of tissue imaging methodology (e.g., ultrasound imaging, X-ray, CT, etc.). FIG. 4B illustrates a cross-section view of a target tissue having a plurality of positioned needle electrodes.

One advantage of methods using the described electrode array or plurality of the present invention is that relative electrode positioning can be directed to smaller distances so as to further allow more precise control of the desired effect of the applied field on the tissue. Factors such as differential conductive properties and resistance or tissue impedance (e.g., differences in muscle, adipose, vasculature, etc.), as well as differential perfusion of blood through vascularized tissue, can limit the ability to control and/or predict effects of delivered current field traversing larger distances through tissue. In the present invention, distances between activated electrodes can be limited to shorter distances, such as a few centimeters or less, for improved control and predictability of current effects (e.g., tissue heating, field delivery, orientation, etc) on the targeted tissue. Thus, activated electrodes in a pair or group can be spaced less than about 4 cm apart. For example, adjacent electrodes of a pair or group will typically be positioned within about 0.1 cm to about 2 cm of each other. Distances of about 0.5 cm have been shown to be particularly effective in providing controlled and predictable field delivery, controlled tissue heating, as well as substantial therapeutic effect.

As described above, a plurality of electrodes can be positioned in the target tissue of a patient and the electrodes can be activated in pairs or groups to deliver the therapeutic current field to destroy cancerous tissue. A particular electrode of an array need not be confined to a single unit, but can be activated at different times in conjunction with different electrodes of the plurality. For example, differential activation can include activating a specific or selected series of electrode groups in a particular or predetermined order. In one embodiment, a series of selected pairs or groups can be activated in seriatim and/or in a predetermined order, with activation control typically being determined by operation or instructions (e.g., programming) of a control system or module. Sequences of group activations can be controlled and repeated, manually or by automation, as necessary to deliver an effective or desired amount of energy.

Such differential activation may advantageously allow delivery of fields throughout the target tissue and in a plurality of different directions. As shown above by way of example, a simple four electrode grouping of an array can be differentially activated in pairs, with each different pair of electrodes providing a different field delivery and orientation (possible field flow/orientations are illustrated by arrows). While activation of electrodes in discrete pairs provides simplicity, electrodes can be activated in groups for more diverse field orientation and deliver. For example, a delivery unit can include a centrally located electrode surrounded by spaced outer or secondary electrodes, with the applied field extending between the central electrode and the outer spaced electrodes. In this manner, the outer electrodes can essentially define an ablation volume with the inner/central electrode positioned within the volume. Field delivery in this way can be controlled and substantially contained within the ablation volume. Examples described herein illustrate electrode positioning including outer electrodes and an inner or centrally located electrode. Electrode positioning will not be limited to any particular configuration, and various arrangements will be possible.

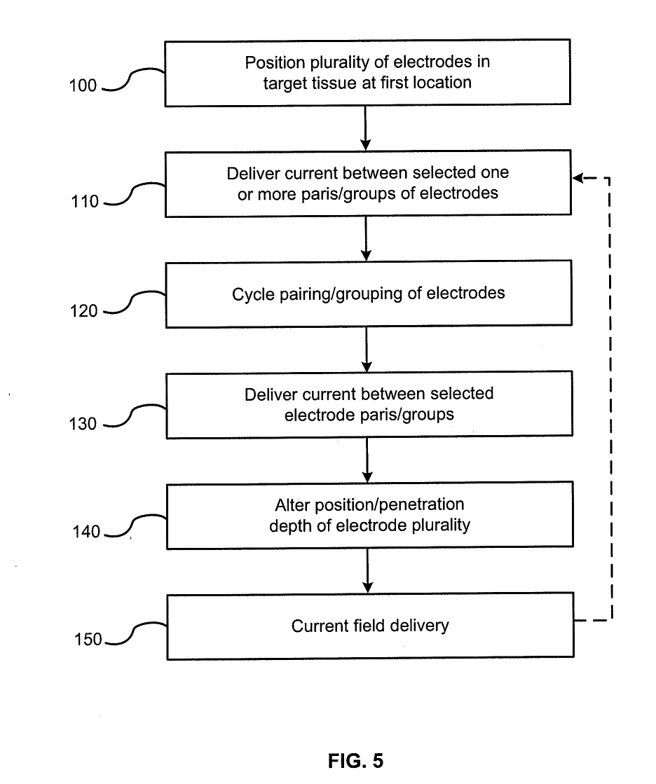

FIG. 5 schematically illustrates a method encompassed by the present invention. As in the embodiments described in FIGS. 3 and 4, for example, a system of the present invention can include a plurality of electrodes (e.g., electrode array) that can be positioned in the target tissue or prostate tissue of a patient, with selected current delivery and application to the tissue occurring by differential activation of various groups or pairs of electrodes. Thus, a method of the present invention, as shown in FIG. 5, can include positioning a plurality of electrodes in a target tissue of a patient at a first or initial treatment location (Step 100). The plurality can be positioned entirely within the patient's tissue or may include positioning of at least some electrodes of the plurality within, at, near, or beyond the target or cancerous tissue margin. In some cases, positioning for initial current delivery may include advancing electrodes through the tissue (e.g., through the skin or entry location) of the patient and to a distal most portion of the target tissue. Electrode advancement and positioning may be aided or guided by tissue imaging techniques. Once the desired initial treatment positioning of the electrodes has been achieved, initial field delivery can occur. As described herein, current can be delivered to the target region of the tissue in a plurality of different directions or current orientations by differentially selecting between and activating different pairs/groups of electrodes. Different groups or pairs of electrodes can be activated individually or in sequence, or a plurality of different groups can be activated simultaneously. For example, treatment can include selecting a first grouping or pairing of electrodes for activation, and delivering current between the selected pairs/groups (Step 110). Current delivery can be cycled through different pairings or groupings of electrodes by discontinuing current delivery through the first selected grouping, and selecting a second or subsequent grouping for activation (Step 120). Following cycling or selecting a different subsequent grouping, current is delivered between the next selected electrode pair/group (Step 130). Following current delivery at the initial treatment positioning of the electrodes, the one or more of the plurality can be removed from the tissue or optionally the position of the electrodes altered for a next phase of current delivery (Step 140). For example, electrodes can be withdrawn a short distance in a proximal direction to alter the electrode penetration depth for a next phase of current field delivery (Step 150). Current delivery and electrode re-positioning may be repeated until the desired volume of the tissue has been treated.

Treatment time according to the present invention can be selected based on a variety of factors, such as characterization of the tissue, energy applications selected, patient characteristics, and the like. Energy application to a target tissue region during treatment according to the present invention can be selected from a few minutes to several hours. In some instances, effective treatment is expected to occur in about 5 minutes to 90 minutes. Effective preferential destruction of cancerous cells has been observed in less than one hour, and in many cases about 15-30 minutes of energy application. Treatment can include a single energy delivery period or dose, or multiple phases or doses of energy application. As described above, electrodes can be positioned in a first location and energy delivered, then moved to subsequent location(s) for subsequent energy delivery. Treatment can occur in phases or repeated, and/or may be coupled with additional or alternative treatments or energy delivery methods.

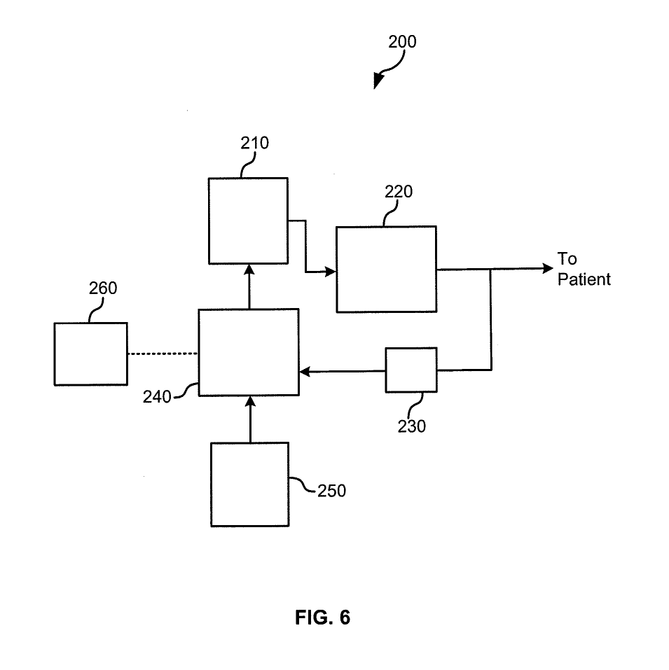

A system according to an embodiment of the present invention is described with reference to FIG. 6. The system 200 can include incorporated therewith any device of the present invention for delivery of energy to the patient, and includes a power unit 210 that delivers energy to a driver unit 220 and than to electrode(s) of an inventive device. The components of the system individually or collectively, or in a combination of components, can comprise an energy source for a system of the invention. A power unit 210 can include any means of generating electrical power used for operating a device of the invention and applying electrical current to a target tissue as described herein. A power unit 210 can include, for example, one or more electrical generators, batteries (e.g., portable battery unit), and the like. One advantage of the systems of the present invention is the low power required for the ablation process. Thus, in one embodiment, a system of the invention can include a portable and/or battery operated device. A feedback unit 230 measures electric field delivery parameters and/or characteristics of the tissue of the target tissue region, measured parameters/characteristics including without limitation current, voltage, impedance, temperature, pH and the like. One or more sensors (e.g., temperature sensor, impedance sensor, thermocouple, etc.) can be included in the system and can be coupled with the device or system and/or separately positioned at or within the patient's tissue. These sensors and/or the feedback unit 230 can be used to monitor or control the delivery of energy to the tissue.

The power unit 210 and/or other components of the system can be driven by a control unit 240, which may be coupled with a user interface 250 for input and/or control, for example, from a technician or physician. The control unit 240 and system 200 can be coupled with an imaging system 260 (see above) for locating and/or characterizing the target tissue region and/or location or positioning the device during use.

A control unit can include a, e.g., a computer or a wide variety of proprietary or commercially available computers or systems having one or more processing structures, a personal computer, and the like, with such systems often comprising data processing hardware and/or software configured to implement any one (or combination of) the method steps described herein. Any software will typically include machine readable code of programming instructions embodied in a tangible media such as a memory, a digital or optical recovering media, optical, electrical, or wireless telemetry signals, or the like, and one or more of these structures may also be used to transmit data and information between components of the system in any wide variety of distributed or centralized signal processing architectures.

Components of the system, including the controller, can be used to control the amount of power or electrical energy delivered to the target tissue. Energy may be delivered in a programmed or pre-determined amount or may begin as an initial setting with modifications to the electric field being made during the energy delivery and ablation process. In one embodiment, for example, the system can deliver energy in a “scanning mode”, where electric field parameters, such as applied voltage and frequency, include delivery across a predetermined range. Feedback mechanisms can be used to monitor the electric field delivery in scanning mode and select from the delivery range parameters optimal for ablation of the tissue being targeted.

Systems and devices of the present invention can, though not necessarily, be used in conjunction with other systems, ablation systems, cancer treatment systems, such as drug delivery, local or systemic delivery, surgery, radiology or nuclear medicine systems, and the like. Another advantage of the present invention, is that treatment does not preclude follow-up treatment with other approaches, including conventional approaches such as surgery and radiation therapy. In some cases, treatment according to the present invention can occur in conjunction or combination with therapies such as chemotherapy. Similarly, devices can be modified to incorporate components and/or aspects of other systems, such as drug delivery systems, including drug delivery needles, electrodes, etc.

It is understood that the examples and embodiments described herein are for illustrative purposes only and that various modifications or changes in light thereof will be suggested to persons skilled in the art and are to be included within the spirit and purview of this application and scope of the appended claims. Numerous different combinations are possible, and such combinations are considered part of the present invention.

What is claimed is:

1. A method of delivering controlled heating to a tissue of a patient, comprising:
positioning a plurality of electrodes in a target tissue region; and
establishing an electrical current flow radially or in a plurality of different directions through a volume of the tissue so as to preferentially ablate cancerous cells in the volume, wherein electrical current flow is established so as to heat the target tissue to an average temperature of about 40-48 degrees C.

2. The method of claim 1, the electrical current flow comprising a alternating current having a frequency of less than about 300 kHz.

3. A method of delivering controlled heating to a target tissue of a patient for preferential destruction of cancerous cells in the target tissue, comprising:
positioning a plurality of electrodes in the target tissue region, the plurality comprising an array of needle electrodes advanced through the patient's tissue and positioned in the target tissue region; and
differentially activating groups of electrodes of the plurality so as to establish an electrical current flow in a plurality of different directions through a volume of the target tissue and preferentially destroy cancerous cells in the volume, wherein electrical current flow is established so as to heat the target tissue to an average temperature of about 40-48 degrees C.

4. The method of claim 3, wherein the positioning comprises advancing electrodes through guides of a template device.

5. The method of claim 3, wherein the differentially activating comprises activating different groups of two or more electrodes of the array in seriatim.

6. The method of claim 3, wherein the electrical current comprises an alternating current flow comprising a frequency of about 50 to about 250 kHz.

7. The method of claim 3, wherein the electrical current comprises an alternating current flow comprising a frequency of about 100 kHz.

8. A system for preferential destruction of cancerous cells of a target tissue of a patient, comprising:
a plurality of electrodes for advancement and positioning in a target tissue region of the patient;
a control system comprising a power source coupled to the electrodes, and a computer readable storage media comprising instructions that, when executed, cause the control system to:
provide electrical current to the electrodes so as to establish a current flow radially or in a plurality of different directions through a volume of the tissue and to preferentially destroy cancerous or hyperplastic cells in the target tissue region;
maintain an average target tissue temperature of less than about 50 degrees C. during energy delivery.

9. A system for preferential destruction of cancerous cells of a target tissue of a patient, comprising:
an array of electrodes for advancement and positioning in a target tissue region of the patient;
a control system comprising a power source coupled to the electrodes, and a computer readable storage media comprising instructions that, when executed, cause the control system to differentially activate groups of electrodes of the plurality so as to establish an electrical current flow in a plurality of different directions through a volume of the target tissue and preferentially destroy cancerous cells in the volume, wherein electrical current flow is established so as to heat the target tissue to an average temperature of about 40-48 degrees C.

10. The system of claim 8, wherein the differentially activating comprises activating different groups of two or more electrodes of the array in seriatim.

11. The system of claim 8, wherein the electrical current comprises an alternating current flow comprising a frequency of less than about 300 kHz and a voltage field of less than about 50 V/cm.

12. A method of delivering an electric field to a target tissue, comprising:
positioning an ablation probe in a tissue of a patient;
deploying a plurality of outer electrodes from the probe to define an ablation volume having a current flow center, wherein deploying an electrode of the plurality comprises deploying a first microtube from a delivery member of the probe along a guide path curving in at least a first direction, wherein curving of the microtube in the first direction directs advancement of the guide distally and away from the current flow center, deploying a second microtube from the first microtube in a second direction distally and substantially parallel to a long axis of the delivery member; and deploying an electrode from a lumen of the second microtube such that the electrode is advanced along an electrode tissue penetration path advancing distally and substantially parallel to the long axis of the delivery member;
positioning an inner electrode substantially within the ablation volume and at the current flow center;
establishing current flow between the inner electrode and outer electrodes and through the ablation volume to provide one or more electric fields extending through the volume in a plurality of different directions, wherein establishing current flow comprises applying an alternating current so as to preferentially destroy cancerous cells within the ablation volume compared to non-cancerous cells within the ablation volume.
